(19) United States
(12) Patent Application Publication
(10) Pub. No.: US 2016/0230202 A1
(43) Pub. Date: Aug. 11, 2016

(54) Process for Generating Synthetic Engineered Recombinant Proteins for Vaccination, Diagnosis and Treatment of Disease

(71) Applicant: Eric William Olle, Kingsport, TN (US)

(72) Inventor: Eric William Olle, Kingsport, TN (US)

(21) Appl. No.: 14/645,342

(22) Filed: Mar. 11, 2015

Related U.S. Application Data
(60) Provisional application No. 61/950,973, filed on Mar. 11, 2014.

(51) Int. Cl. C12P 21/00; C07K 14/495; C07K 14/535; C07K 14/54

(52) U.S. Cl. CPC C12P 21/00; C07K 14/5406; C07K 14/5418; C07K 14/495; C07K 14/535; C07K 2319/40; C07K 2319/60; C07K 2319/50

(57) ABSTRACT

A process for generating a synthetic engineered recombinant protein has been performed by providing an original protein design and the necessary probabilistic priors for random in silico assembly. Once the in silico synthetic protein is assayed using mathematic modeling, the proteins are then reverse translated into DNA. The DNA fragment, modifying the original DNA to obtain a first DNA fragment, wherein the modifying step comprising adding an antigen optimization sequence to the original DNA fragment, purifying the first DNA fragment, performing a DNA self-assembly or a ligation, adding an amplification linker to obtain a second DNA fragment, purifying the second DNA fragment, transferring the second DNA fragment into an expression vector, expressing the second DNA fragment into a synthetic engineered recombinant protein, as well as purifying the synthetic engineered recombinant protein.

The current application claims a priority to the U.S. Provisional Patent Application Ser. No. 61/950,973, filed on Mar. 11, 2014.

### FIELD OF THE INVENTION

The present invention relates generally to disease treatment and vaccination. More specifically, the present invention is a process for generation of synthetic engineered recombinant proteins for vaccination, diagnosis and treatment of disease.

### BACKGROUND OF THE INVENTION

It is known that in many cases, in order to obtain sufficient expression of an exogenous gene or artificial sequence, certain regions in gene sequence, which may affect in vivo gene expression, have to be enhanced, attenuated or altered with respective to the specific organism in which the gene sequence is expected to be expressed. Nevertheless, the design of an optimized DNA sequence for expression is not an easy approach. It is quite labor consuming and time consuming as well. Therefore, it is in an urgent need of a functional and convenient process or program to help researchers and companies to quickly obtain an optimized DNA sequence for further expression or other applications.

In light of the foregoing, one objective of the present invention is to provide a process for generating synthetic recombinant DNA sequences that are optimized for gene expression in one or more organisms this method starts at the level of a “in silico probabilistic protein” where the probability of an amino acid(s) or protein fragment(s). Further, in addition to incorporation of highly optimized and engineered recombinant DNA sequences for desirable expression, the present invention also provides a novel technique that can be utilized for generation of any potential antigen for immune system or a mixture of antigens. Moreover, the present invention demonstrated potential “linker” regions designed to aid the Synthetic Engineered Recombinant Protein (SyERP) to be processed into appropriate protein fragments to be presented to immune cells etc in vivo. In addition, another aspect of the present invention is the use of multiple existing protein domains to allow for tertiary structure of the SyERP, as well as the use of protein-protein interaction domains that can be added to the SyERP to allow for a higher order protein structure as part of a multi-ordered engineered biological system.

This invention has the potential to assemble high number of amino acid, peptide or protein fragment information for the stimulation of the immune system or generation of any potential protein depending on the initial assumptions of the researchers. For example, using the higher order antigenic protein with each arm of 75 kDa (to minimize glomerular filtration if it breaks apart) it may be possible to assemble greater than 50 different vaccines against a multitude of organisms in one higher order protein. For example, a 148 kDa protein has been designed to decrease the autoimmune production of anti-B-cell antibodies.

There is more than one type of SyERP that can be used for applications outside immune regulation. Additional applications are the linking to extracellular matrix (ECM) binding regions and several growth factors and inhibitors. Additionally, attachment and chemoattractant signals can be added along with different collection of matrix metalloproteinase (MMP) or other protease consensus sequences to either mask or expose additional factors.

The linkage of a fluorescent protein arms to the higher order proteins allows for a potential diagnostic application. If one “arm” of a multimer SyERP is a fluorescent protein (i.e. GFP or similar) an ansitropy using polarization filters sent perpendicular and correct excitation/emission spectral reading. In brief, Brownian motion sets up a random rotation of the SyERP that if bound by another complex, or cut apart by an enzyme. This change in mass will change the force the Brownian motion acts and then change the velocity of the SyERP. This can be a complex to other florescent assays to multiplex complex cellular reactions on a single engineered protein system.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

FIG. 1 is a representation of Synthetic Engineered Recombinant Proteins (SyERP).

FIG. 2 is a diagram of potential recombination or fusion protein.

FIG. 3 is a diagram of method for generation of Synthetic Engineered Recombinant Proteins (SyERP).

FIG. 4 depicts full length SyERP designed to be used as part of a single protein strand or a higher order SyERP.

### DETAIL DESCRIPTIONS OF THE INVENTION

All illustrations and embodiments are for the purpose of describing selected versions of the present invention and are not intended to limit the scope of the present invention.

In the present invention, multiple proteins are used for initial description, to demonstrate that the techniques outlined in the document are applicable to a plurality of target proteins or mixtures of targets that can be used in a range of downstream applications. In addition to presenting a novel protein engineering technique, the present invention also shows how multiple approaches can be applied to generate SyERPs.

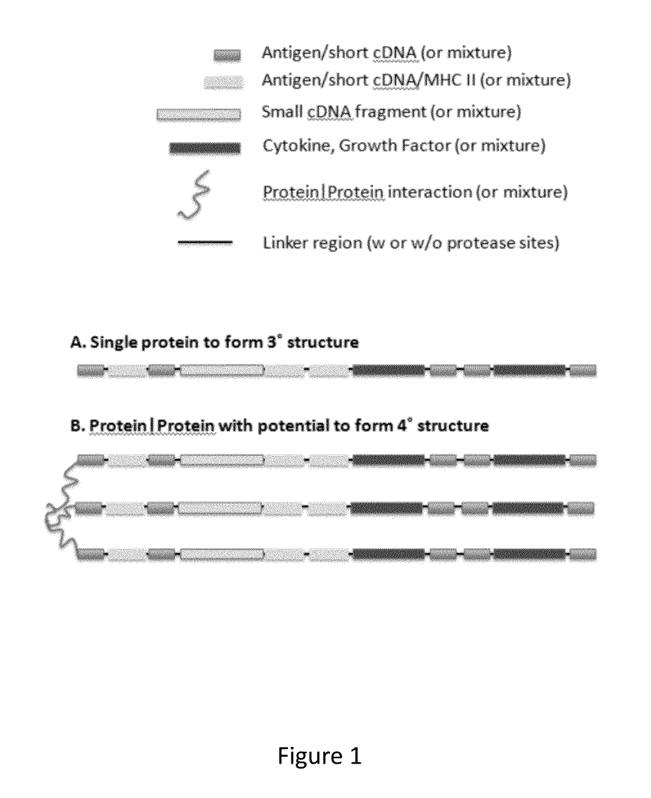

In reference to FIG. 1, a simple illustration of Synthetic Engineered Recombinant Proteins (SyERP) has been demonstrated. In FIG. 1, a diagram of potential sub domains use in a protein designed to stimulate based upon on or more small fragments of the protein from MHC to sub cloned regions of the protein. These can be fused or engineered with specific protein domains to modify functions (i.e. glycosylation, targeting to organelles; etc.) linker is a short region of recombinant DNA designed to produce appropriate linker proteins for specific targets. For example, MMP-9 and Cathepsin D may be used for antigen optimization. Additional section that can be used are simple [Gly-X-Y]n repeats to allow for the trimerization of proteins in a higher order structure. It is noted that the use of a collagen or lectin type repeat is only for the purpose of description.

As mentioned above, the basic concept of the present invention is shown in FIG. 1. It includes both the single and higher order protein structures. One goal of this invention is shown in FIG. 1 and is a combination of one or more proteins for the activation or suppression of immune response and/or other cellular functions. In the basic diagram, the single protein does not have an interacting area or if the protein is placed into a system that allows for protein, protein interaction, or a 4° structure. Diagramed are potential ways of basic assembly, however, when used to activate or repress immune function, it would be composed of the appropriate antigen or mixture of antigens. The antigens can be a mixture of more than one potential molecular target, such as PSA; CCEA; LR and p53, or multiple regions from the same gene. In addition, these synthetic structures can be fused with one or more cytokines, chemokines, growth factors or others. They may include linker regions or may be directly fused to one another. While in FIG. 1, it has shown that the same protein is replicated across three arms, there could be three different proteins or and combination thereof as long as the protein maintains its downstream functions.

As an example of multiple downstream functions, B-cells may be stimulated by fusing IL-4, IL-7, and/or TGF-beta along with multiple linked repeats of the known activating antigen. As for dendritic cell stimulation, it would be possible to fuse with GM-CSF; IL-1beta along with engineered antigens for optimal expression and use in a clinical setting. Another way to stimulate dendritic cells would be to use: C3b, C3g/d, C3 or C3d CR2 binding region fused to antigens or antigenic repeats along with an immunoglobin or similar domain. Additional B-Cell activation may occur by adding immune-modulators to the complement fusion proteins. Finally using higher order systems one can describe several combinations across two or more interacting proteins. The higher order proteins could also be fused with an engineered GFP or other fluorescent marker that could be determine changes in the rotation (anisotropy moment) and potential binding or removal or any item that would modify the Brownian motion induced rotation of the molecule.

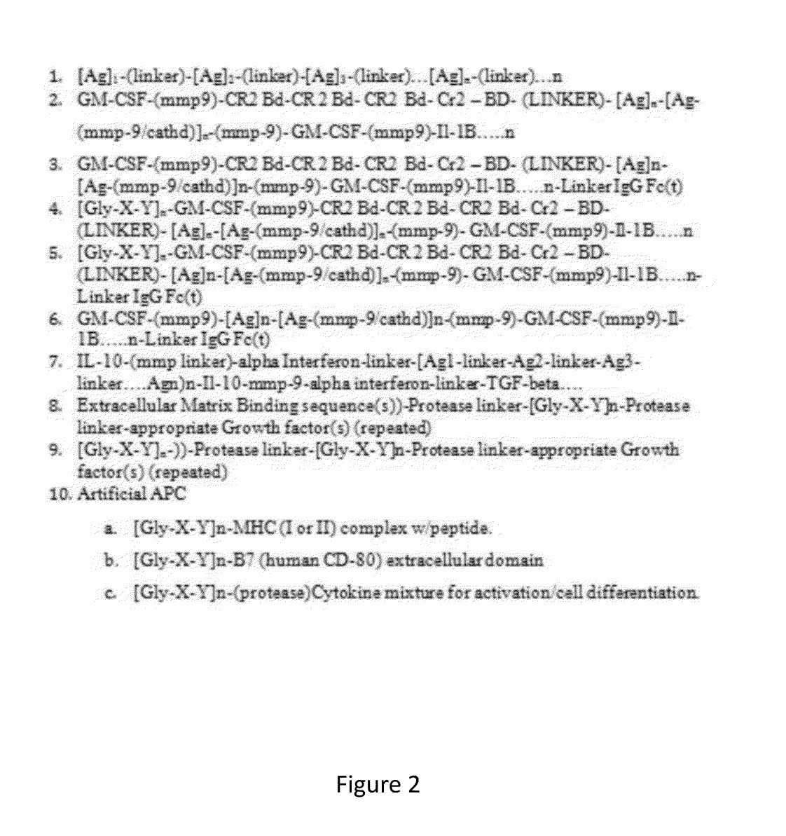

In reference to FIG. 2, it has shown a diagram of potential combinations/fusion proteins. It is noted that this is not meant to be an exhaustive list but highlights a few of the potential synthetic proteins that can be created. The majority is for immune activation, immune suppression, cell growth and differentiation. Generally speaking, there are currently multiple way to achieve the aim of the present invention, which includes oligonucleotide self assembly; micro dsDNA ligation; GeneArt™ strings from Invitrogen Inc. They are shown with specific examples using the Laminin Receptor and other proteins. For example, the laminin receptor protein was run through an antigen analysis program (K & T 1990) to provide a list of the following antigens that were reversely translated into cDNA segments using optimized E. coli codon bias (see FIG. 2). When the SyERP [Ag-Protease]n DNA sequence is compared against the entire database of nucleotide sequences there were no significant matches. Establishing that this a novel protein.

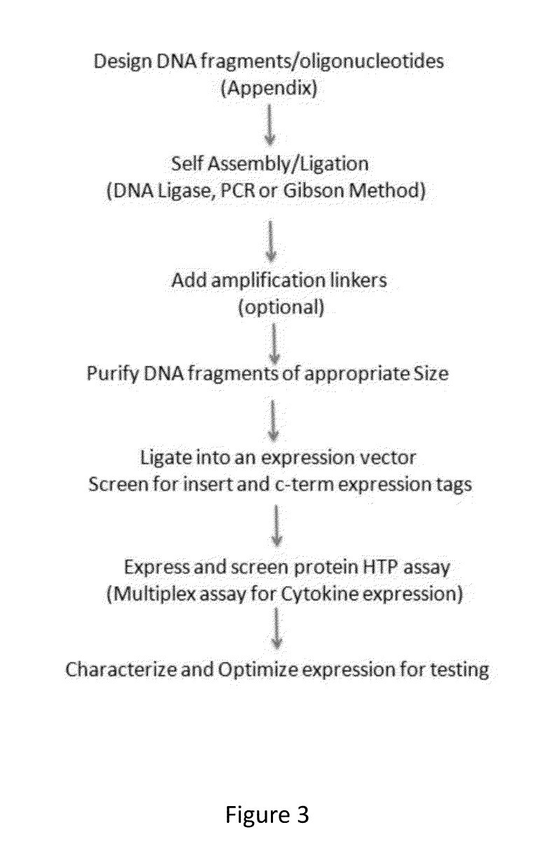

Further, as shown in FIG. 3, there are multiple methods for assembly and manufacturing SyERPs a few are listed here include but not limited to: oligonucleotide assembly (A), short segments of double stranded DNA (B), longer strands of DS/SS DNA (or RNA) (C), GeneArt™ strings and combinations thereof (D) and finally in silico/computer methods (not shown). Sequences used to generate oligonucleotides or nucleic acid polymers are shown in Appendix 1.

In order to enhance immune activation, addition of C3d Cr2 binding region and/or the IgG Fc regions were done to show the complexity of the engineered proteins. Due to the fact antibodies are often glycosylated, the protein sequences may be reversely translated into other organisms. Any codon look-up table can be used (i.e Saccharomyces spp., viral or higher organism expression systems) this allows for addition of glycosylation and other post-translational modifications not commonly done in prokaryotic recombinant expression systems.

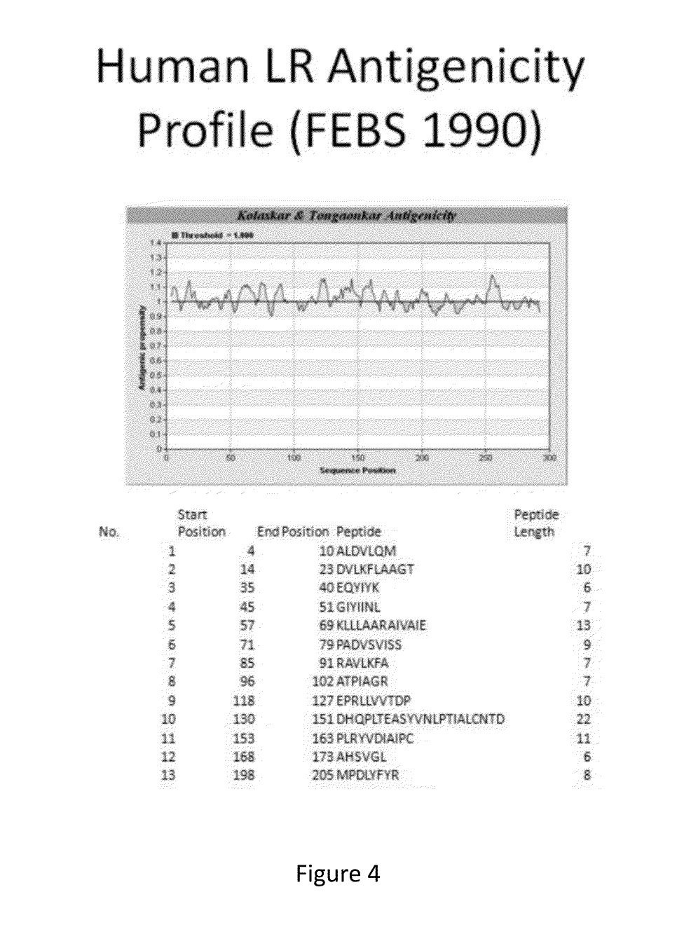

FIG. 4 shows a diagram of Full length SyERP designed to be used as part of a single protein strand or a higher order SyERP. The first part shows three different DNA sequences. In part A (i), this is a simple conglomeration of Laminin Receptor antigenic sites as determined using the Kolaskar and Tongaonkar (1990) method randomly assembled with MMP-9 and/or Cathepesin D protease sites located in-between. Similar to part A(i) part (ii) use LR with Protease sites and interspersed immune modulators the next par (iii) use a combination of more than one potential antigenic protein source (i.e. LR, PSA, CCEA, transferrin or any protein antigen) interspersed (or alone) with the immune modulators. A gene search of the database revealed no significant similarities except for short segments of proteins or the immunomodulator factors. When the use of the protein sequence to reverses translate an engineered cDNA and add protease/fusion sites would reduce nucleotide identity at or below 85%. This is a significant reduction of identity at the nucleotide level with the exact same protein sequence since gaps will often reduce identity score.

The second part shows how a collagen/lectin trimerization repeat [Gly-X-Y]ₙ can be added as part of the SyERP or part of a premade DNA vector and the above are cloned into to allow for higher order assembly of proteins. Other options for four individual arm would be to clone a cringle region of Immunoglobulin or LAG-3 with the disulfide bridges and then generate and purify in the presence of BME or DTT to reduce the sulfide bonds. When ready to assemble dialyze to lower the sulfhydryl reducing agents below critical concentration one removed, the protein can be allowed to refold if purified under denaturing conditions (i.e. Guandidium HCl, Urea etc). Similar methods can be used to “piggy-back” or protect proteins on other higher order SyERPs.

This system can be modified in multiple ways and shows some of the potentials associated with engineered proteins. There are several ways to assemble the SyERPs to activate DC and just a few options are described as a way of assembling an antigen, antibody (or Fc region) and C3d-CR2 interaction region. First, assume a simple system of just the antigens and multimers of the C3d-CR2 region. Next the addition of the Fc region, followed by a collared collagen repeats with cytokine for higher organization synthetic proteins. This system can either be designed for high throughput screening methods using a range of different assays. Briefly, after one or more oligo's are selected an appropriate linker regions are added this allow for single stranded synthesis. It may be possible to assemble SyERPs using a three-oligo system with one oligo being bridge. Once assembled and ligated the synthetic DNA at this point can have added amplification Tags on 5′ and 3′ ends to allow for additional enrichment of longer/lower probability events.

Once evaluated for correct length of engineered DNA construct, it is ligated into an expression system that allows for rapid screening of the presence of an insert (b-galactosidase) and a carboxy-end epitope tag to allow for rapid screening/enrichment for recombinant inserts. Purifying the plasmid can further screen recombinant bacterial colonies with DNA inserts, verifying insert and expression in a cell free system that is screened for appropriate responses. The same method can be used to create synthetic proteins fused to one or more cytokines; for example GM-CSF and IL-1beta may be fused alongside smaller, but more informative antigenic regions (to down regulate, IL-10 may be fused instead). For further enrichment, the B and T cell receptor complement can be analyzed using standard methods and the SyERP can be further refined for genetic and/or individual differences. There may come a time where proteins are synthesized specifically for the genetic background of the patient or the expression pattern of the diseased tissue to maximize clinical response while minimizing non-specific reactions that may lead to vaccine related public outcry.

How can a synthetic protein be made to stimulate monocytes, dendritic cells to endocytosis and process antigens? A simple fusion molecule using the previously deduced Fc region amino acid sequence on the amino end of the Fc(t) region, a multiple repeat of antigens along with cytokines can be placed. This recombinant protein can be purified in the presence of DTT or sulfhydryl reducing agent and then removed the reducing agents to allow for the disulfide bond forms between two Fc regions.

Additional protein structures, cytokines, antigens, complement proteins can be placed on a light chain clone that contains the disulfide bonds found in native immunoglobulins. A potential SyERP sequence composed of laminin receptor antigens, Fc(trypsin) and CR2 binding domain repeats. Once a SyERP is generated on the protein level (or DNA/RNA) it may be optimized first.

Some of the potential optimizations that can include (but not limited to):

DNA Level

### 1. Codon Optimization

2. Removal of cryptic stop codons
3. Addition/removal of restriction enzyme sites
4. Addition/removal of DNA binding protein elements

### RNA Level

1. Addition/Removal of RNA stability factors
2. Addition/Removal of secondary Structure
3. Addition/Removal of translational start/stop sequences
4. Addition/Removal of poly A+ tail

### 5. Addition/Removal of 5′ and 3′ UTR

### Protein Level (Design Level)

1. Addition/Removal of protein sub-domain building blocks (i.e. kringle domains; lectin binding domains; carbohydrate binding domains; Protein|Protein interaction domains; Growth Factors; Cytokine, chemokines, immune regulators, cell binding domains, Immunoglobulin domains, LAG-3 domains, b-Sheet, Turns, A-helix etc).
2. Addition/Removal of Protease sites

### 3. Addition/Removal of Glycosylation Sites

### 4. Addition/Removal of Phosphorylation Sites

5. Addition/Removal of regulatory domains
6. Addition/Removal of Disulfide bonds

### 7. Addition/Removal of Acetylation

Once optimized, the protein can SyERP can be Reverse Translated to generate a novel RT-DNA. Unlike cDNA, or complementary DNA that requires an RNA, SyERPS are designed at the protein level and does not require RNA for generation. For example, when using fusions of small antigenic regions separated by protease sites and fused to one or more cytokines two interesting things happen. First, on the nucleotide level there is no significant similarities found; however on the protein level, significant similarities are seen in the cytokine domains. Because the cytokines are only a small part of the total SyERP, there is very little to no homology to any protein seen and by definition this is novel.

Downstream applications include dendritic, T-cell or B-cell Therapy. Current vaccine technology is unmatched in its use of either peptides or longer length cDNA fragments. The peptides are chemically produced and are fairly standardized in the approach but due to technological constraints limited to antigens less than 50 amino acids and these peptides must be injected with adjuvant or conjugated to a larger carrier protein (i.e. KLH). This can be used for basic repeats and micro-synthetic proteins, however the small size of peptides limits blood half life due to kidney filtration and may require a charged design to bind serum albumin or other common hapten binding proteins. Fusion proteins and cDNA fragments used in vaccines and other therapies are useful but often have excessive non-informative protein sequences that can lead to potential activation of the immune system in a way that may cause increasing side effects and autoimmune profiles of self-antigens. In addition to generation of rtDNA's for protein production, the DNA and RNA of the SyERP itself may be used to elicit a cellular response. Therefore, a new way to present specific antigens to monocytes and dendritic cells is needed to further the current immunotherapy field. I

The goal of the current SyERP design in this section is to fuse to multiple protein domains to allow for increased presentation to monocytes and dendritic cells when compared to full-length cDNA or peptides. Several different constructs can be used listed below is a basic description for several different options. While this may be different for each organism etc it is still possible. Option 1, fusion to random antigen repeats separated by protease sites using one or more cytokines. This protein in its basic form is composed of one or immunomodulatory factor and antigenic repeats of one or more region, protein or both. Finally, small linker regions containing protease sites have been created. In its simplest form may just be a long repeat of antigens. For example say we wanted to create a monocyte/dendritic cell SyERP what would it look like? This needs to be consider in context of Bayesian approach and genetic algorithms that can generate the sum of all plausible outcomes. This make SyERPS a probabalist protein that is then designe in silico, translated and then screened for function. One example may be:

[Ag]1-(protease)-[Ag]2-(protease)-[Ag]3-(protease) . . . [Ag]n-(protease) . . . n

In the diagram above, [Ag] 1, 2, or 3 can be the same or different and may even combine more than one antigen from more than one protein. In addition to protein-protein interactions for generation higher order proteins, chemicals such as Polyethylene glycol branches, Dextran polymers, starch polymers, zymosan may act a substrates/scaffolding combinations of the different SyERBs and/or cell modulatory factors. It is also possible to add additional domain such as glycosylation signals, sub cellular localization signals, phosphorylation signals, protease digest sites and conformational structure to aid in the presentation or use of SyERPs.

Option 2. Fusion to immunomodulator protein domains to increase antigen presentation. For example:
GM-CSF-(mmp9)-CR2 Bd-CR 2 Bd-CR2 Bd-Cr2-BD-(LINKER)-[Ag]n-[Ag-(mmp-9/cathd)]n-(mmp-9)-GM-CSF-(mmp9)-Il-1B . . . n
Option 3. Higher combination of Options 1 and 2.
GM-CSF-(mmp9)-CR2 Bd-CR 2 Bd-CR2 Bd-Cr2-BD-(LINKER)-[Ag]n-[Ag-(mmp-9/cathd)]n-(mmp-9)-GM-CSF-(mmp9)-Il-1B . . . n-Linker IgG Fc(t)
or

### [Gly-X-y]n-GM-CSF-(mmp9)-CR2 Bd-CR 2 Bd-CR2 Bd-Cr2-BD-(LINKER)-[Ag]n-[Ag-(mmp-9/cathd)]n-(mmp-9)-GM-CSF-(mmp9)-Il-1B . . . n

or

[Gly-X-y]n-GM-CSF-(mmp9)-CR2 Bd-CR 2 Bd-CR2 Bd-Cr2-BD-(LINKER)-[Ag]n-[Ag-(mmp-9/cathd)]n-(mmp-9)-GM-CSF-(mmp9)-Il-1B . . . n-Linker IgG Fc(t) plus (3-fold collagen repeat x two-fold IgG Fc(t) disulfide bonds)

GM-CSF-(mmp9)-[Ag]n-[Ag-(mmp-9/cathd)]n-(mmp-9)-GM-CSF-(mmp9)-Il-1B . . . n-Linker IgG Fc(t)

Autoimmune response is common in most patients and the self-recognizing B-Cell may sit in a state of stasis. This should be considered a way to “anti-vaccinate” the individual by forcing the auto-immune cells to enter anergy, die or generally become unresponsive to the auto-immune epitopes. Anti-ColIVa epitopes are fusion proteins of potential epitopes fused to down regulator cytokines. These are fusion proteins of potential epitopes fused to IL-10, IL-10 & alpha interferon, IL-10 & TGF-beta or any combination: (Note: more than one protein antigenic regions (blue and green), more than one cytokine (Black) and universal MMP linker (yellow). Additionally, it is not uncommon for circulation if IgM auto-immune antibodies but due to their size rarely induce the full auto-immune response. Therefore, if a B-Cell can be forced to enter anergy or apoptosis and/or an IgG reaction is minimized patients may be able to have excellent quality of life. Similar to above for the formation of the antigens for vaccination or dendritic cell sensitization but fused with immune down regulator (IL-10; IL-4, TGF-beta, IL-4 alpha-interferon or similar combinations to induce anergy/immune down regulation). However, to increase efficacy in cell death/deactivation it may be necessary to fuse with mutated/modified forms to decrease binding and potentially increase probability of cell death, down regulator with Fc binding region mutants and/or Cr2 mutants. In this example two potential options are shown. First, is a simple fusion of IL-10 to anti-glomerular basement membrane (anti-GBM) epitopes from the alpha 3 collagen IV.

The second example uses the multiple major antibody epitopic regions that are correlated to more aggressive forms of diabetes. The initial demonstration shows two proteins that are correlated to pancreatic cell destruction, however, it is easy to add insulin and other proteins that generate autoimmune antibodies in progression of diabetes. This is fused with both IL-10 and C3d/CR2 binding domain mutants to decrease the probability of active IgG immune response. This could easily be expanded on include the insulin and other antibody epitopes to allow for both decrease in pancreatic damage and maintenance of insulin action with out binding by circulating antibodies.

These down regulator proteins, or anti-vaccines, could have important implications in the treatment of potential side effects due to immunotherapy or in allergy treatment. As immunotherapy use increases and we develop increasing number of self-antigens for therapeutic use, the probability of developing an autoimmune reaction increases, therefore, an anti-immunotherapy is needed for correction of potential side effects. If a patient develops a side effect to a developed immunotherapy, the initial response maybe anti-inflammatory steroids followed by an injection of IL-10 containing SyERPs with the proper antigens or with mutated antigens that can compete for the sites to prevent activation while increased IL-10 release or fusion to apoptotic signals decrease the number of active epitope specific cells.

Using a similar method to multiple antigenic responses can be used in a similar method for tissue engineering and 3d organ printing or other methods. Multiple ECM binding protein exists and can be used for example a laminin binding protein from bacterial was fused to chemoattractant and FGF proteins. (A). In part B, a collagen repeat [Gly-X-Y]n is used to allow for co-polymerization with the ECM. After the ECM anchor section, a series of either cryptic or specific proteases can be used. In addition, know the regions of the ECM and cells interact, engineered sequences can be created to mask until the correct time. A potential diagram is show:

Extracellular Matrix Binding sequence(s))-Protease linker-[Gly-X-Y]n-Protease linker-appropriate Growth factor(s) (repeated), or

{Gly-X-Y]n-))-Protease linker-[Gly-X-Y]n-Protease linker-appropriate Growth factor(s) (repeated

Two different potentials are shown in the extracellular matrix binding protein SyERP's depending on the protein sequence may be possible to bind laminin elastin, collagen etc. This current invention is being shown as a way for either: vascular growth, Fibroblastic growth/chemoattraction, or smooth muscle laydown. This is meant as a simple example that can be expanded upon. In the first example, ECM binding sequence can be one of many deduced peptides that bind to elastin, fibronectin, collagen, laminin and/or other proteins. Some of the deduced laminin receptor and laminin binding sequences have specific properties that allow for use in a ECM printing system that could be used to print the next generation of artificial surgical mesh, standardized cadaver skin replacement, and almost any type of engineered organ. Once the ECM is printed, it can be store for later use either hydrated or desiccated for hydration by the clinician (i.e. biological mesh, skin alternative etc.). In more complex systems, for example engineered replacement organs, the ECM can be designed and standardized cell line or patients cells allowed to grow onto the multi-dimensional ECM. How would this work?

The following is meant to be a basic example and not the only way to use SyERPs in ECM printing applications: The following diagram shows a basic idea for a hybrid Biological-Synthetic mesh designed for a couple of possibilities. First, a removable substrate that can be used in vivo is placed onto a tray and wetted/treated with appropriate substrates to allow for binding of the ECM to the substrate (unless substrate is just a carrier for the ECM then no treatment needed). The tray is temperature controlled at low temp and flooded with a solution of ECM (i.e. collagen, laminin, elastin or mixtures) to just barely over the top of the substrate (similar to sterolitography). If there is a need for regions that do not bind cells two potential options: (i) Deglycosylated the laminin/ECM or (ii) place a competitive inhibitor. If there is a need to increase growth etc., and in a laminin solution, a SyERP with laminin binding sequences contains appropriate growth factors or chemo attractant peptide sequences that have been previously deduced. This SyERP will need to be designed to have appropriate affinity for the ECM to allow for appropriate localization without too much diffusion while no inhibiting the expected cellular phenotype by binding to the same regions of the ECM. There are several way to integrate the SyERPs into the ECM is co-localize as the ECM is being printed. Printing can occur one of many ways but one is to have a cooled solution on a platen an use a laser, microwave, radio wave etc. to increase the local temp to the point that the protein polymerizes. The other option is to spray in to a pool of warmed buffer (contain the SyERP) similar to an ink-jet printer. The mechanism of placing the ECM with SyERP is only summarized.

Previous work on creating artificial APC/dendritic cells has concentrated on a substrate (i.e. small beads) this removes the need to assemble on a substrate and allows for stepwise self-assembly. This assembly can further be organized in a lipid micelle or solid substrate for increased efficacy.
1. [Gly-X-Y]n-MHC (I or II) complex w/peptide. Using MHC clones from previously sensitized dendritic cells or another option would be to sub clone the individuals TCR, BCR or MHC II.
2. [Gly-X-Y]n-B7 (human CD-80) extracellular domain
3. [Gly-X-Y]n-(protease)Cytokine mixture for activation/cell differentiation.

The three molecules are generated independent and stored under denatured or slightly acidic conditions so not to allow the collagen repeat tails to interact until ready to stimulate naïve T-Cells either in vivo, ex vivo, or in situ. An example of use as artificial dendritic cells follows standard Adoptive Immunotherapy methods and procedures with slight modifications. Briefly, to determine potential SyERP-APC MHCII and MHCI proteins will be modified to contain a collagen repeat and minimize protease sites that may decrease efficacy. Next a similar clone will be made of CD80 and finally a cytokine mixture that is designed for the type of expected response. If attempting to

A potential option for the higher order proteins is to allow for interaction with a fluorescent protein (i.e. GFP or eq) with a collagen repeat; this module is allowed to interact with either a protein that will be reduced by proteases increasing the rotational moment. On the other hand, if looking for binding of another protein, change in the rotational moment to a slower protein using fluorescent anisotropy/polarization to calculation apparent rotation.

The above is meant to be a basic learning on SyERPs and there are untold numbers of different possibilities from targeting transcription factors that are engineered for specific activation with appropriate fail-safe measures. One could imagine an engineered protein that would activate apoptosis if and only if the Jak/Stat, Nfkb, p53 and other signal transduction pathways are disrupted. Another possibility for a higher order engineered protein would be to create a protease system that would stay inactive until in the microenvironment of: clotting factors (stroke); atherosclerotic plaques, neurofibrillary tangles or other accumulation of items that can be enzymatically removed.

The future of protein engineering has barely been started; Initial work has used the framework of full-length cDNA's cloned from PCR products and potentially fused to other sub-cloned regions to generate this early form of chimera proteins. These inventions show that using prior art of early-published protein sequences that can be reverse translated into an engineered cDNA (see LR patent) that has serious implications on in silico design and optimization for all standardized expression systems. The next step is to design proteins from the ground up for specific biological function that may or may not look like the original proteins. A prime example is the use of SyERPs for the generation of micro RT-cDNA sequences that are linked together to generate a truly novel protein. This protein can be generated by a computer program to screen to fit user defined parameters.

The Workflow for SyERP generation is described below in details:

1. Read or Generate protein fragments (Provided by user can be from individual amino acid to full protein fragments)
2. Read or generate protease fragments or other protein segments (part of linker regions/conjunctions)
a. Note: May want to include a random range: Poisson, uniform, binomial or other forms of probability modeling
3. Rate and/or cull fragments using (Some assumption for molecular mass)
4. Read or generate SYERP
a. NOTE may want to include a dual random assembly process to allow multiple ratios
5. Read or generate protein rules (optional)
6. Read or generate conjunctions (turns, cys sect, c3d, IgG, CSF)
7. Work to generate an effective “ideal” syerp to evaluate the generated population
8. Run a normalize functions as appropriate on individual components
9. Assemble the first generations using either uniform, normal or log norm for distributions and normalized p values for other added items and/or weighting using Shannon type Information content modification.
10. Measure fitness on a range of protein information
a. Distance between cys
b. Distance between other
c. Number of runs of X or less allowed w/o a protease site for the same “region”
d. pH
e. pI
f. length
g. p representation of each
h. p each is a proper site
i. Expected target characteristics
j. Size
k. Protein frag numbers
l. Shannon Information code or other mathematical modeling
m. Hamming code with p of each word
11. Visually describe the population and then look at to verify
12. Cluster and assign cluster distance breeding p's for complex groups
13. Define the Ploidy model (1, 2 or 2n)
14. Random combination base on plausibility or other accepted Bayesian/Genetic Algorithm
15. Randomly assign potential offspring fitness that is used to randomly calculate number of off spring for a combined
16. Allow for culling, random mutation etc.
17. Repeat until certain fitness is obtained. Run cluster analysis to see different groups clustering round specific attributes and/or combination of attributes.
18. Allow for population crashes and/or regional cross breeding if one population get lager/smaller.
19. After predefined fitness is obtained either store
20. Monitor for bloat (GA known “problem”) via standard mathematical models (i.e. modify Ward Decision theory and/or combine Min Entropy and Max Entropy or signal/noise inferences to provide a Bayesian MCMC simulation for max and minimum length predicted for the amount of prior information provided.

Although the invention has been explained in relation to its preferred embodiment, it is to be understood that many other possible modifications and variations can be made without departing from the spirit and scope of the invention.

What is claimed is:

1. A process for generating a synthetic engineered recombinant protein, comprising
providing an original DNA fragment;
modifying the original DNA to obtain a first DNA fragment, wherein the modifying step comprising adding an antigen optimization sequence to the original DNA fragment;
purifying the first DNA fragment;
performing a DNA self-assembly or a ligation;
adding an amplification linker to obtain a second DNA fragment;
purifying the second DNA fragment;
transferring the second DNA fragment into an expression vector;
expressing the second DNA fragment into a synthetic engineered recombinant protein; and
purifying the synthetic engineered recombinant protein.

2. The process for generating a synthetic engineered recombinant protein as claimed in claim 1, comprising
the antigen optimization sequence is a MMP-9 DNA sequence.

3. The process for generating a synthetic engineered recombinant protein as claimed in claim 1, comprising
the antigen optimization sequence is a Cathepsin D DNA sequence.

4. The process for generating a synthetic engineered recombinant protein as claimed in claim 1, comprising
the modifying step comprising adding a DNA sequence encoding a [Gly-X-Y]n repeat to the original DNA fragment, wherein the [Gly-X-Y]n repeat facilitates to form a trimerization structure of the synthetic engineered recombinant protein, X is an amino acid, Y is an amino acid, and n is more than 2 and less than 500.

5. The process for generating a synthetic engineered recombinant protein as claimed in claim 1, comprising
the modifying step comprising adding a DNA sequence selected from the group consisting of cytokine, chemokine and growth factor, and adding a linker sequence.

6. The process for generating a synthetic engineered recombinant protein as claimed in claim 5, comprising
the cytokine is selected from the group consisting of IL-4, IL-7, and TGF-beta, wherein the cytokine stimulates B cells.

7. The process for generating a synthetic engineered recombinant protein as claimed in claim 5, comprising
the cytokine is GM-CSF, wherein the cytokine stimulates dendritic cells.

8. The process for generating a synthetic engineered recombinant protein as claimed in claim 1, comprising
the modifying step comprising adding a DNA sequence encoding a fluorescent marker, wherein the fluorescent marker is used to determine a change in the synthetic engineered recombinant protein's Brownian motion.

9. The process for generating a synthetic engineered recombinant protein as claimed in claim 1, comprising
the step of purifying the synthetic engineered recombinant protein comprising purifying in the presence of a sulfhydryl reducing agent of BME or DTT to reduce sulfide bonds, dialyzing to remove the sulfhydryl reducing agent, and allowing the protein to refold.

10. The process for generating a synthetic engineered recombinant protein as claimed in claim 1, further comprising a step of optimization at DNA level, wherein the optimization at DNA level selected from the group consisting of codon optimization, removal of cryptic stop codon, addition of restriction enzyme site, remove of restriction enzyme site, addition of DNA binding protein element, removal of DNA binding protein element, and a combination thereof.

11. The process for generating a synthetic engineered recombinant protein as claimed in claim 1, further comprising a step of optimization at RNA level, wherein the optimization at RNA level selected from the group consisting of addition of RNA stability factor, removal of RNA stability factor, addition of translational starting sequence, removal of translational starting sequence, addition of translational stop sequence, removal of translational stop sequence, addition of poly A tail, removal of poly A tail, addition of 5′ and 3′ UTR, removal of 5′ and 3′ UTR, and a combination thereof.

12. The process for generating a synthetic engineered recombinant protein as claimed in claim 1, further comprising a step of optimization at protein level, wherein the optimization at protein level selected from the group consisting of, addition of a protein sub-domain, removal of a protein sub-domain, addition of a protease site, removal of a protease site, addition of a glycosylation site, removal of a glycosylation site, addition of a phosphorylation site, removal of a phosphorylation site, addition of a disulfide bond, removal of a disulfide bond, addition of an acetylation bond, removal of an acetylation bond, and a combination thereof.

13. The process for generating a synthetic engineered recombinant protein as claimed in claim 1, further comprising a step of providing a chemical substance that functions as a substrate or scaffold for the synthetic engineered recombinant protein, wherein the chemical substance is selected from the group consisting of polyethylene glycol (PEG), dextran polymers, starch polymer, zymosan and a combination thereof.

14. A process for generating a synthetic engineered recombinant protein, comprising
providing an original DNA fragment;
modifying the original DNA to obtain a first DNA fragment, wherein the modifying step comprising adding an antigen optimization sequence to the original DNA fragment, and the antigen optimization sequence is a MMP-9 DNA sequence or a Cathepsin D DNA sequence;
purifying the first DNA fragment;
performing a DNA self-assembly or a ligation;
adding an amplification linker to obtain a second DNA fragment;
purifying the second DNA fragment;
transferring the second DNA fragment into an expression vector;
expressing the second DNA fragment into a synthetic engineered recombinant protein;
purifying the synthetic engineered recombinant protein;
optimizing at DNA level, wherein the optimization at DNA level selected from the group consisting of codon optimization, removal of cryptic stop codon, addition of restriction enzyme site, remove of restriction enzyme site, addition of DNA binding protein element, removal of DNA binding protein element, and a combination thereof;
optimizing at RNA level, wherein the optimization at RNA level selected from the group consisting of addition of RNA stability factor, removal of RNA stability factor, addition of translational starting sequence, removal of translational starting sequence, addition of translational stop sequence, removal of translational stop sequence, addition of poly A tail, removal of poly A tail, addition of 5′ and 3′ UTR, removal of 5′ and 3′ UTR, and a combination thereof;
optimizating at protein level selected from the group consisting of, addition of a protein sub-domain, removal of a protein sub-domain, addition of a protease site, removal of a protease site, addition of a glycosylation site, removal of a glycosylation site, addition of a phosphorylation site, removal of a phosphorylation site, addition of a disulfide bond, removal of a disulfide bond, addition of an acetylation bond, removal of an acetylation bond, and a combination thereof; and
providing a chemical substance that functions as a substrate or scaffold for the synthetic engineered recombinant protein, wherein the chemical substance is selected from the group consisting of polyethylene glycol (PEG), dextran polymers, starch polymer, zymosan and a combination thereof.

15. The process for generating a synthetic engineered recombinant protein as claimed in claim 14, comprising
the modifying step comprising adding a DNA sequence encoding a [Gly-X-Y]n repeat to the original DNA fragment, wherein the [Gly-X-Y]n repeat facilitates to form a trimerization structure of the synthetic engineered recombinant protein, X is an amino acid, Y is an amino acid, and n is more than 2 and less than 500.

16. The process for generating a synthetic engineered recombinant protein as claimed in claim 14, comprising
the modifying step comprising adding a DNA sequence selected from the group consisting of cytokine, chemokine and growth factor, and adding a linker sequence.

17. The process for generating a synthetic engineered recombinant protein as claimed in claim 16, comprising
the cytokine is selected from the group consisting of IL-4, IL-7, and TGF-beta, wherein the cytokine stimulates B cells.

18. The process for generating a synthetic engineered recombinant protein as claimed in claim 16, comprising
the cytokine is GM-CSF, wherein the cytokine stimulates dendritic cells.

19. The process for generating a synthetic engineered recombinant protein as claimed in claim 14, comprising
the modifying step comprising adding a DNA sequence encoding a fluorescent marker, wherein the fluorescent marker is used to determine a change in the synthetic engineered recombinant protein's Brownian motion.

20. The process for generating a synthetic engineered recombinant protein as claimed in claim 14, comprising
the step of purifying the synthetic engineered recombinant protein comprising purifying in the presence of a sulfhydryl reducing agent of BME or DTT to reduce sulfide bonds, dialyzing to remove the sulfhydryl reducing agent, and allowing the protein to refold.
